(19) United States
(12) Patent Application Publication
(10) Pub. No.: US 2026/0166125 A1
(43) Pub. Date: Jun. 18, 2026

(54) NLRP12 AND NLRC5 MODULATORS AND METHODS FOR USING THE SAME TO MODULATE DISEASES

(71) Applicant: St. Jude Children's Research Hospital, Inc., Memphis, TN (US)

(72) Inventors: Thirumala-Devi KANNEGANTI, Memphis, TN (US); Balamurugan SUNDARAM, Memphis, TN (US)

(21) Appl. No.: 19/123,619

(22) PCT Filed: Oct. 26, 2023
(86) PCT No.: PCT/US2023/077823

§ 371 Date: Apr. 23, 2025

Related U.S. Application Data
(60) Provisional application No. 63/422,601, filed on Nov. 4, 2022.
(60) Provisional application No. 63/501,430, filed on May 11, 2023.

(51) Int. Cl. A61K 38/19; A61K 38/42; A61K 45/06; A61P 7/04; A61P 29/00; G01N 33/50; G01N 33/68

(52) U.S. Cl. CPC A61K 38/191; A61K 38/42; A61K 45/06; A61P 7/04; A61P 29/00; G01N 33/5041; G01N 33/6869; G01N 33/6872; G01N 2333/4703; G01N 2333/545

(57) ABSTRACT

Disclosed are compositions and methods for using heme and at least one PAMP and/or DAMP molecule for activating NLRP12 and/or NLRC5 and inflammatory cell death. Also provided is a screening assay for identifying NLRP12 and/or NLRC5 inhibitors and use of such inhibitors in the treating or ameliorating of NLRP12-mediated or NLRC5-mediated inflammation associated with a hemolytic disease, infectious disease, cancer, or inflammatory syndrome.

### INTRODUCTION

This application claims benefit from U.S. Provisional Patent Application Serial Nos. 63/422,601, filed Nov. 4, 2022 and 63/501,430, filed May 11, 2023, the contents of which are incorporated herein by reference in their entireties.

This invention was made with government support under grant nos. AI101935, AI124346, AI160179, AR056296, and CA253095 awarded by the National Institutes of Health. The government has certain rights in this invention.

### STATEMENT REGARDING ELECTRONIC FILING OF A SEQUENCE LISTING

A Sequence Listing in XML text format, submitted under 37 C.F.R. § 1.821-1.834, entitled “SJ0105WO_ST26,” 6,280 bytes in size, generated Sep. 18, 2023, and filed via EFS-Web, is provided in lieu of a paper copy. This Sequence Listing is hereby incorporated by reference into the specification.

### BACKGROUND

Innate immune sensors called pattern recognition receptors (PRRs) play a key role in protecting the host from invading pathogens. Each sensor recognizes specific pathogen-associated molecular patterns (PAMPs) or endogenous damage-associated molecular patterns (DAMPs; which include cellular components such as proteins, nucleic acids, or lipids, as well as cytokines and alarmins) to activate its cognate innate immune signaling pathways, which include nuclear factor-κB (NF-κB), mitogen activated protein kinase (MAPK) and type I interferon (IFN) pathways. One sub-family of cytosolic PRRs called nucleotide-binding oligomerization domain (NOD)-like receptors (NLRs) play diverse roles in activating inflammasomes and cell death as well as inflammation. While a few NLRs are relatively well-characterized, little is known about the functions of many other sensors in this family.

While engagement of PRRs, including NLRs, is often beneficial and can reduce pathogen burden in the infected host, excess activation can also lead to pathogenic inflammation, cytokine storms, tissue damage and DAMP release. Together, PAMPs and DAMPs released during infections and inflammatory conditions can further induce multi-organ failure and mortality. However, the innate immune sensors involved in detecting the collective release of PAMPs and DAMPs and their role in activating inflammasomes and inflammatory cell death to contribute to disease pathogenesis remain poorly defined.

NLR Family Pyrin Domain Containing 12 (NLRP12, also known as RNO, NALP12, PYPAF7, and Monarch-1) is a pyrin-containing NLR protein. Human NLRP12 is expressed predominantly in cells of myeloid lineage, such as neutrophils, eosinophils, monocytes, macrophages, and immature dendritic cells. Mutations in the NLRP12 gene have been associated with a class of autoinflammatory syndromes, called NLRP12AD, which include some forms of familial cold autoinflammatory syndrome (Jeru et al. (2008) Proc. Natl. Acad. Sci. USA 105:1614-1619). Studies have shown how the nonsense mutation p.Arg284X, which is located within the NBD of NLRP12, is less effective in suppressing NF-κB activity compared with the wild-type NLRP12 (Borghini et al. (2011) Arthritis Rheum. 63:830-839). In line with a loss of function causing inflammation, an insertion generating a splicing defect also induces a clear reduction of the inhibitory properties of NLRP12 on NF-κB signaling. In contrast to these findings, however, the missense mutation p.Asp294Glu, mapping within the evolutionarily conserved NBD, associates with an increased caspase-1 activation rather than with inhibition of NF-κB signaling (Jeru et al. (2011) Arthritis Rheum. 63, 1459-1464).

NLRP12 has also been implicated as an inflammasome component, recognizing Yersinia pestis, the causative agent of plague. Nlrp12^{-/-} mice showed higher mortality and bacterial load after Y. pestis infection, where the NLRP12 inflammasome was shown to be a central regulator of IL-18 and IL-1β production, mediated by caspase-1 activation. Furthermore, NLRP12 also induces IFN-γ production via IL-18; however, Nlrp12^{-/-} had minimal effect on NF-κB signaling after infection with Y. pestis strains (Vladimer et al. (2012) Immunity 37:96-107). In addition, the presence of NLRP3 and NLRP12 in inflammasome complexes in monocytes derived from malaria patients, as well as in mouse models, has led to the suggestion that the NLRP3/NLRP12-dependent activation of caspase-1 is likely to be the key event regulating the systemic expression of IL-1β and mediating the hypersensitivity of malaria patients to secondary bacterial infections (Ataide et al. (2014) PLoS Pathog. 10:e1003885). However, the specific ligands activating NLRP12 and the downstream signaling pathways involved in these contexts are unknown.

Similar to NLRP12, NLR Family Caspase Activation and Recruitment Domain Containing 5 (NLRC5, also known as NOD27 and CLR16.1) is also an NLR protein. Human NLRC5 is expressed in many cellular lineages, including immune cells (T and B cells, myeloid cells) as well as microglial cells, gastric secreting cells, and adipocytes, among others. Missense mutations in human NLRC5 have been associated with neurological conditions, including schizophrenia and amyotrophic lateral sclerosis. However, whether these mutations are causative remains to be determined. NLRC5 is predominantly known for its role as a transcriptional regulator of MHC-I expression. In inflammation, contrasting functions for NLRC5 have been reported. Under some conditions, NLRC5 is thought to negatively regulate inflammation by reducing NF-κB and type I IFN signaling (Cui et al. (2010) Cell 141:483-496), while under other conditions NLRC5 contributes to inflammation by promoting NLRP3 inflammasome activation and cytokine release (Davis et al. (2011) J. Immunol. 186:1333-1337; Kumar et al. (2011) J. Immunol. 186:994-1000), but each of these phenotypes is only observed in specific cell types and under specific conditions. Overall, the function of NLRC5 and its roles in inflammation remain unclear.

### SUMMARY OF THE INVENTION

This invention is a composition for inducing the production of or activating NLRP12 or NLRC5, wherein said composition is composed of heme and at least one PAMP molecule and/or DAMP molecule. In certain aspects, the at least one PAMP molecule comprises a triacylated lipoprotein, lipoteichoic acid, peptidoglycan, porin, zymosan, Pam₃CSK₄, diacylated lipopeptide, dsRNA, polyadenylic-polyuridylic acid, polyinosinic:polycytidylic acid, lipopolysaccharide, flagellin, single-stranded RNA, CpGA, Poly G10, Poly G3, CpG oligonucleotides, PamCysPamSK₄, Toxoplasma gondii profilin, double-stranded RNA, 5′ppp-dsRNA, phosphorylcholine, lipoarabinomannan, mycolic acid, β-1,3-glucan, N-formylmethionine, mannose-rich glycan, CL307, imiquimod, gardiquimod, resiquimod, motolimod, UC-IV150, EMD120108, IMO-2125, VTS-1463GS-9620, GSK2245035, TMX-101, TMX-201, TMX-202, isatoribine, AZD8848, MEDI9197, 3M-051, 3M-852, 3M-052, 3M-854A, S-34240, KU34B, CL663, SB9200, SB11285, or 8-substituted 2-amino-3H-benzo[b]azepine-4-carbozamide. In other aspects, the at least one DAMP molecule is tumor necrosis factor α or heme metabolites such as biliverdin. A pharmaceutical composition including the heme and at least one PAMP and/or DAMP molecule in admixture with a pharmaceutically acceptable carrier, excipient, vehicle, diluent, or preservative is also provided.

The invention also includes methods for inducing inflammatory cell death and treating cancer or other disease or condition that would benefit from inflammatory cell death by administering to a subject in need of such treatment heme and at least one PAMP and/or DAMP molecule. In some aspects, the other disease or condition is an infection, proliferative condition or condition comprising damaged cells.

Further provided is a method for treating or ameliorating NLRP12-mediated or NLRC5-mediated inflammation associated with a hemolytic disease, infectious disease, inflammatory syndrome, or cancer by administering to a subject in need thereof an effective amount of an inhibitor of NLRP12 or NLRC5 activation thereby treating or ameliorating the subject's NLRP12-mediated or NLRC5-mediated inflammation associated with the hemolytic disease, infectious disease, inflammatory syndrome, or cancer. In some aspects, the inhibitor of NLRP12 or NLRC5 production is a small molecule, peptide, antisense oligonucleotide, guide RNA, shRNA, antibody or an antibody fragment that targets upstream regulatory molecules of NLRP12 or NLRC5 including, e.g., Toll-like receptors (TLRs), reactive oxygen species (ROS), the nicotinamide pathway, and/or interferon regulatory factors (IRFs). In other aspects, the inhibitor of NLRP12 or NLRC5 activation is a small molecule, peptide, antisense oligonucleotide, guide RNA, shRNA, antibody or an antibody fragment that directly inhibits NLRP12 or NLRC5 activation (e.g., directly interacts with NLRP12 or NLRC5 or nucleic acids encoding NLRP12 or NLRC5). In other aspects, the hemolytic disease is beta thalassemia, hemolytic anemia, or sickle cell disease; the infectious disease is SARS-CoV-2, malaria, influenza, or pneumonia; and the inflammatory syndrome is neurological inflammation or systemic inflammatory response syndrome.

A method for identifying an agent that inhibits NLRP12-dependent or NLRC5-dependent inflammasome and inflammatory signaling activation and inflammatory cell death is also provided. This invention involves the steps of (a) contacting a sample of cells with heme and at least one PAMP or DAMP molecule to induce production of or activate NLRP12 or NLRC5; and (b) contacting the sample of cells of (a) with at least one test agent, wherein a decrease in the production or activation of NLRP12 or NLRC5 in the presence of the test agent as compared to the production or activation of NLRP12 or NLRC5 in the absence of the test agent is indicative of an agent that inhibits NLRP12-dependent or NLRC5-dependent inflammasome and inflammatory signaling activation and inflammatory cell death. In some aspects, production or activation of NLRP12 or NLRC5 is determined by measuring the mRNA or protein amounts of NLRP12 or NLRC5; the cleavage of one or more of gasdermin D, gasdermin E, caspase-1, caspase-8, caspase-3, caspase-7; phosphorylation of mixed-lineage kinase domain-like protein; cell death; or release of IL-1β or IL-18 by the cells of the sample.

### BRIEF DESCRIPTION OF THE DRAWINGS

FIG. 1 shows the quantification of cell death in wild-type (WT) bone marrow-derived macrophages (BMDMs) in response to media or treatment with combinations of Pam3CSK4 (Pam3) with LPS, poly(I:C) or R848; or LPS with poly(I:C) or R848; or poly(I:C) with R848 for 48 hours. Data are representative of at least three independent experiments. ****P<0.0001. Analysis was performed using the one-way ANOVA. Data are shown as mean±SEM.

FIG. 2 shows the quantification of cell death in WT BMDMs treated with HMGB1, MSU, heme or S100A8/A9 in combination with PAMPs for 48 hours. Data are representative of at least three independent experiments. ****P<0.0001. Analysis was performed using the one-way ANOVA. Data are shown as mean±SEM.

FIG. 3 shows the quantification of cell death in WT and Nlrp12^{-/-} bone marrow-derived macrophages (BMDMs) treated with heme and Pam3, heme and LPS, or heme and R848 for 36 hours or heme and TNF-α for 48 hours. Data are representative of at least three independent experiments. ****P<0.0001. Analysis was performed using the unpaired t test. Data are shown as mean±SEM.

FIG. 4 shows the quantification of cell death in WT and Nlrc5^{-/-} bone marrow-derived macrophages (BMDMs) treated with heme and Pam3, heme and LPS, or heme and TNF-α for 48 hours. Data are representative of at least three independent experiments. ****P<0.0001. Analysis was performed using the unpaired t test. Data are shown as mean±SEM.

FIG. 5 shows the quantification of cell death in WT and Casp1^{-/-}Casp8^{-/-}Ripk3^{-/-} (TKO) bone marrow-derived macrophages (BMDMs) stimulated with heme and Pam3 or heme and LPS for 36 hours. Data are representative of at least three independent experiments. ****P<0.0001. Analysis was performed using the unpaired t test. Data are shown as mean±SEM.

FIG. 6 shows the quantification of ASC specks in WT and Nlrp12^{-/-} BMDMs treated with heme and Pam3 for 36 hours. Unstimulated WT BMDMs were used as a mock control. Data are representative of at least three independent experiments. ****P<0.0001. Analysis was performed using the one-way ANOVA. Data are shown as mean±SEM.

FIG. 7 shows measurements of IL-1β and IL-18 release from the supernatant of WT and Nlrp12^{-/-} BMDMs treated with heme and Pam3 for 42 hours. Data are representative of at least three independent experiments. *P<0.05. Analysis was performed using the unpaired t test. Data are shown as mean±SEM.

FIG. 8 shows the relative expression of Nlrp12 and Nlrc5 mRNAs in WT BMDMs treated with media, heme, Pam3 or the combination of heme and Pam3 for 36 hours. Data are representative of at least three independent experiments. ****P<0.0001. Analysis was performed using the one-way ANOVA. Data are shown as mean±SEM.

FIG. 9 shows serum BUN release in WT mice injected with PBS (n=5), LPS (n=5), phenylhydrazine (PHZ; n=6) or LPS and PHZ (n=6). Data are representative of at least three independent experiments. ****P<0.0001. Analysis was performed using the one-way ANOVA. Data are shown as mean±SEM.

FIG. 10 shows serum creatinine release in WT and Nlrp12^{-/-} mice or Nlrc5^{-/-} injected with PBS or LPS and PHZ (WT PBS, n=5-9; WT LPS+PHZ, n=8-17; Nlrp12^{-/-} LPS+PHZ, n=7; Nlrc5^{-/-} LPS+PHZ, n=[n=18]). Data are representative of at least three independent experiments. *P<0.05, ****P<0.0001. Analysis was performed using the one-way ANOVA. Data are shown as mean±SEM.

FIG. 11 shows survival in WT, Nlrp12^{-/-}, and Nlrc51 mice injected with LPS and PHZ. Data are representative of at least three independent experiments. **P<0.01, ****P<0.0001. Analysis was performed using the log-rank test (Mantel-Cox) test.

FIG. 12 shows survival in WT and Nlrp12^{-/-}Nlrc5^{-/-} mice infected with Plasmodium berghei ANKA (1×10⁵ infected red blood cells). ****P<0.0001. Analysis was performed using the log-rank test (Mantel-Cox) test.

FIGS. 13A-13B illustrate the activation of NLRP12-mediated (FIG. 13A) and NLRC5-mediated (FIG. 13B) cell death and inflammation.

### DETAILED DESCRIPTION OF THE INVENTION

Innate immunity provides the first line of defense against infection and sterile insults. Innate immune sensors are critical to assemble cytosolic protein complexes, such as inflammasomes, that induce inflammation and inflammatory cell death to clear infectious agents and alert the broader immune system. NLRP12 and NLRC5, cytosolic innate immune sensors, have been associated with several infectious and inflammatory diseases. However, their roles in inflammasome activation and inflammatory cell death, as well as the specific triggers that activate them, remain unknown.

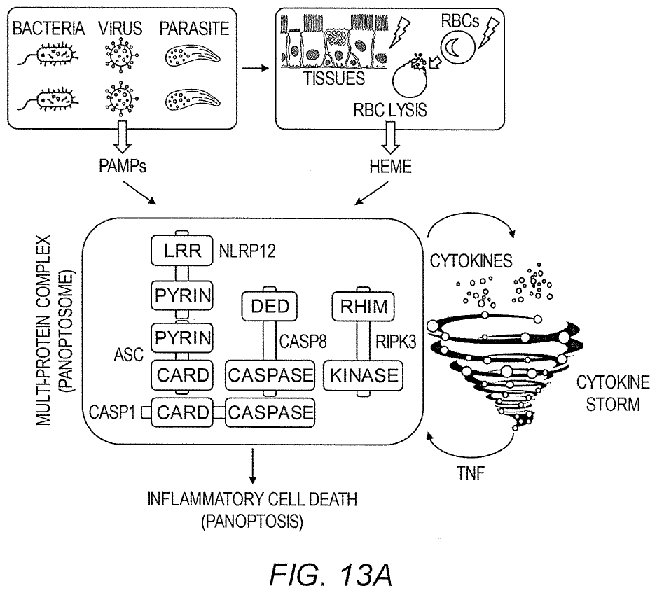

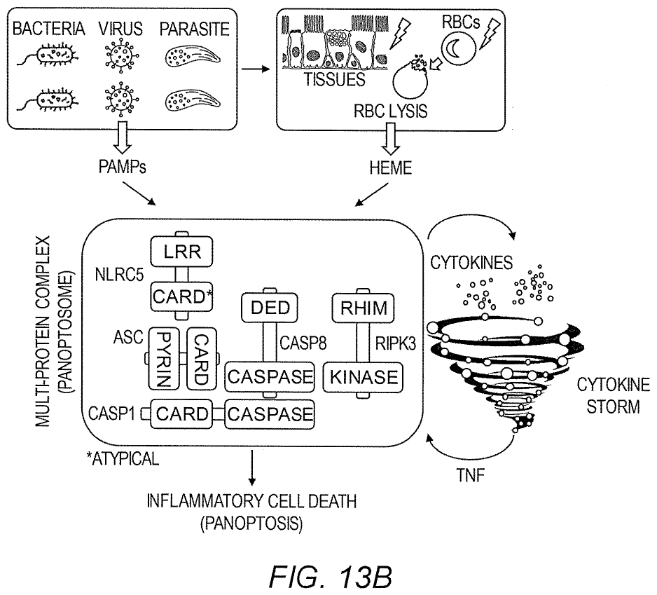

It has now been found that NLRP12 and NLRC5 both activate caspase-1 to drive IL-1β and IL-18 maturation and induces inflammatory cell death through the caspase-1/caspase-8/RIPK3 axis (FIGS. 13A-13B). In particular, it was observed that a composition composed of heme plus PAMPs or DAMPs mimics infection to induce inflammatory cell death. In addition, it was shown that NLRP12 and NLRC5 are the innate immune cytosolic sensors responsible for the inflammasome activation and inflammatory cell death in response to heme and PAMPs or DAMPs. In patients, NLRP12 was highly upregulated across multiple infections and inflammatory conditions, including SARS-CoV-2, influenza, pneumonia, systemic inflammatory response syndrome (SIRS) and hemolytic diseases. Moreover, deletion of Nlrp12 or Nlrc5 significantly protected mice from mortality in a hemolytic model, and combined deletion of Nlrp12 and Nlrc5 significantly protected mice from mortality in a hemolytic infection model. Overall, NLRP12 and NLRC5 were shown to be essential cytosolic sensors for heme-mediated inflammasome activation, inflammatory cell death and pathology, indicating that NLRP12 and NLRC5 and inflammatory cell death molecules can be drug targets for hemolytic diseases and inflammatory syndromes. More specifically, production and activation of NLRP12 or NLRC5 could be therapeutically beneficial to clear infections, damaged cells, or tumor cells, whereas blockade of NLRP12 or NLRC5 activation and the downstream cell death pathway can be used to prevent inflammation during hemolytic and infectious diseases, inflammatory syndromes and cancers.

Thus, in accordance with the present invention, the expression or activity of NLRP12 and/or NLRC5 may be modulated (i.e., activated or inhibited) using one or a combination of modulators (i.e., activators or inhibitors). The term “activator” is not intended to embrace non-selective inducers of all gene expression or protein synthesis. Likewise, the term “inhibitor” is not intended to embrace non-selective suppressors of all gene expression or protein synthesis, or general toxins.

In one aspect, the present invention provides a composition composed of heme and at least one PAMP and/or DAMP molecule and use of the same in activating NLRP12 and/or NLRC5 and inducing inflammatory cell death. Advantageously, the combination of heme and at least one PAMP and/or DAMP molecule provides a synergistic increase (i.e., more than additive increase) in inflammatory cell death making this combination useful in clearing infections, damaged cells, or tumor cells.

NLR Family, Pyrin Domain-Containing 12 (NLRP12), also known as Nacht Domain-, Leucine-Rich Repeat-, and Pyd-Containing Protein 12 (NALP12); Pyrin Domain-Containing Apaf1-Like Protein 7 (PYPAF7); Regulated by Nitric Oxide (RNO) and Monarch-1, has an N-terminal pyrin domain (PYD), followed by a FISNA (fish-specific NACHT-associated domain), NACHT domain, a NACHT-associated domain (NAD), and a C-terminal leucine-rich repeat (LRR) region. NLRP12 functions as a negative regulator of TLR- and TNFR-induced NF-κB signaling in context-dependent manners in human cells. NLRP12 blocks IRAK (IL-1R-associated kinase)-1 hyperphosphorylation/activation and facilitates the degradation of NF-κB-inducing kinase (NIK), leading to reduced NF-κB activation. The amino acid sequence of human NLRP12 is known in the art and available under UNIPROT Accession No. P59046.

NLR Family, Caspase Activation and Recruitment Domain-Containing 5 (NLRC5), also known as NOD27 and CLR16.1, has an N-terminal atypical caspase activation and recruitment domain (CARD), followed by a NACHT domain, a NACHT-associated domain (NAD), and a C-terminal leucine-rich repeat (LRR) region. NLRC5 functions remain largely unknown, but roles have been suggested as a transcriptional regulator of MHC-I and as both a positive and negative regulator of inflammation through NF-κB and IFN signaling, as well as NLRP3 inflammasome regulation. The amino acid sequence of human NLRC5 is known in the art and available under UNIPROT Accession No. Q86W13.

For the purposes of this invention, “heme” or “hemin” refers to protoporphyrin IX containing a ferric iron (Fe³⁺) ion with a coordinating chloride ligand.

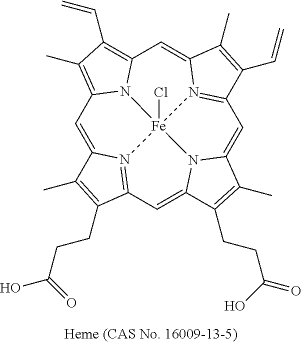

Heme may be obtained and isolated to homogeneity from natural sources or synthesized. Exemplary commercial sources of heme include Thermo Scientific, Sigma-Aldrich, and Selleckchem.

The compositions and methods of this invention include at least one PAMP and/or DAMP molecule in combination with heme. In some aspects, the compositions and methods may include two, three, four or more PAMPs and/or DAMPs. As used herein, “Pathogen-associated molecular pattern molecules” or “PAMPs” are microbial molecules that share a number of different general “patterns,” or structures, that alert immune cells to destroy intruding pathogens. It is well established that PAMPs are recognized by pattern recognition receptors (PRRs), such as Toll-like receptors (TLRs), nucleotide-binding oligomerization domain-like receptors (NLRs), and the mannose receptor expressed on innate immune cells. See, e.g., Amarante-Mendes et al. (2018) Front. Immunol. 9:2379.

As used herein, “Damage-associated molecular pattern molecules” or “DAMPs” are components released by dead, dying, or damaged cells that share a number of different general “patterns,” or structures, that alert immune cells by interacting with PRRs. Although DAMPs contribute to the host's defense, they promote pathological inflammatory responses. DAMPs of use in this invention are recognized by macrophages or other cell types, and inflammatory responses are triggered by different pathways, including TLRs, inflammasomes and PANoptosomes. DAMPs can originate from different sources and include extracellular proteins, such as biglycan and tenascin C; intracellular proteins, such as high-mobility group box 1 (HMGB1), histones, S100 proteins, heat-shock proteins (HSPs); and plasma proteins, like fibrinogen, Gc-globulin, and serum amyloid A (SAA). In addition to proteins, DAMPs can include nucleic acids, or lipids, as well as cytokines and alarmins. In particular aspects, the DAMPs of use in this invention include, but are not limited to, tumor necrosis factor α (TNF-α) or heme metabolites such as biliverdin.

Examples of PAMPs of use in this invention include, but are not limited to, triacylated lipoprotein, lipoteichoic acid (CAS No. 56411-57-5), peptidoglycan, porin, zymosan (CAS No. 58856-93-2), Pam₃CSK₄ (CAS No. 112208-00-1), diacylated lipopeptide, dsRNA, polyadenylic-polyuridylic acid (Poly A:U, e.g., CAS No. 24936-38-7), polyinosinic:polycytidylic acid (Poly I:C; e.g., CAS No. 42424-50-0), lipopolysaccharide (LPS), flagellin, single-stranded RNA (ssRNA), CpGA, Poly G10, Poly G3, CpG oligonucleotides, PamCysPamSK₄, Toxoplasma gondii profilin, double-stranded RNA (dsRNA), 5′ppp-dsRNA, phosphorylcholine (CAS No. 107-73-3), lipoarabinomannan, mycolic acid (CAS No. 37281-34-8), β-1,3-glucan, N-formylmethionine (CAS No. 4289-98-9), and mannose-rich glycan (i.e., a short carbohydrate chain with the sugar mannose or fructose as the terminal sugar). In certain aspects, the PAMP is Pam₃CSK₄ or LPS.

The LPS and/or porin of use in this invention may be obtained from the outer membrane of a Gram-negative bacterial cell wall; the peptidoglycan and/or lipoteichoic acid may be obtained from the cell wall of a Gram-positive bacterium; the lipoarabinomannan and/or mycolic acid may be obtained from the cell walls of an acid-fast bacterium; the zymosan is from yeast cell walls; phosphorylcholine and other lipids are obtained from microbial membranes; and the dsRNA and ssRNA may be obtained from viruses or synthetically produced. As is known in the art, CpG oligonucleotides are unmethylated cytosine-guanine oligonucleotide sequences that are found at a high frequency in the genomes of bacteria and viruses.

Peptidoglycan molecules are characterized as containing meso-diaminopimelic acid (meso-DAP), an amino acid that is unique to peptidoglycans. PAMPs can be obtained from natural sources (e.g., bacterial cell walls such as from Bacillus Calmette-Guerin (BCG) or viral genomes), or synthesized (e.g., Pam₃CSK₄ or PamCysPamSK₄).

Other molecules that are recognized by pattern recognition receptors and may be used in accordance with the present invention include CL307 (CAS No. 1548551-79-6), imiquimod (CAS No. 99011-02-6), gardiquimod (CAS No. 1020412-43-4), resiquimod (R848; CAS No. 144875-48-9), motolimod (CAS No. 926927-61-9), UC-IV150, EMD120108, IMO-2125, VTS-1463GS-9620, GSK2245035, TMX-101, TMX-201, TMX-202, isatoribine, AZD8848, MED19197, 3M-051, 3M-852, 3M-052, 3M-854A, S-34240, KU34B, CL663, SB9200, SB11285, and 8-substituted 2-amino-3H-benzo[b]azepine-4-carbozamide. See also Gambara et al. (2013) J. Cell. Mol. Med. 17(6):713-722. In certain aspects, the PAMP is Resiquimod (R848).

Compositions including heme and at least one PAMP and/or DAMP molecule find particular use in activating NLRP12 and/or NLRC5, activating inflammasomes and/or other multiprotein complexes (such as PANoptosomes), and/or inducing inflammatory cell death (PANoptosis), e.g., to clear infections, damaged cells, or tumor cells. In particular, coadministration of heme and at least one PAMP and/or DAMP molecule provides a synergistic increase in inflammatory cell death. Therefore, this invention provides methods for activating NLRP12 and/or NLRC5 and activating inflammatory cell death by contacting cells with an effective amount of a composition including heme and at least one PAMP and/or DAMP molecule. Cells that may be treated in accordance with this method include populations of cells, in particular population of cells including undesirable cells such as infected cells or tumor cells. As used herein, an “effective amount” is an amount of a substance sufficient to effect beneficial or desired results. In accordance with this method of the invention, the desired result is production and activation of NLRP12 and/or NLRC5 or inflammatory cell death. Production and activation of NLRP12 and/or NLRC5 can be determined by measuring the expression (e.g., transcript or protein) or activity of NLRP12 and/or NLRC5 (e.g., modulation of downstream signaling pathways). Inflammatory cell death can be assessed as described herein by, e.g., propidium iodide incorporation.

In some aspects, the invention provides for the administration of heme and at least one PAMP and/or DAMP molecule to a subject having, suspected of having, or at risk of having cancer or other disease or condition that would benefit from inflammatory cell death, e.g., a proliferative disease, infected cell or condition including or mediated by damaged cells, in order to treat or ameliorate the symptoms of the cancer, proliferative disease, infection, or other disease or condition. The heme and at least one PAMP and/or DAMP molecule are administered in an effective amount to effect treatment or reduction or mitigation of the cancer or other disease or condition. In some aspects, therapy is initiated after the appearance of clinical signs and/or symptoms of cancer. In other aspects, the treatment or reduction or mitigation of the cancer or other disease or condition is affected by the administration of the heme and at least one PAMP and/or DAMP molecule in the absence of any other therapeutic agent.

Whether administered before or after the development of clinical signs and/or symptoms of cancer, administration of heme and at least one PAMP and/or DAMP molecule can reduce the signs or symptoms of cancer. In this respect, an effective amount of the heme and at least one PAMP and/or DAMP molecule is an amount that can, e.g., reduce or inhibit tumor growth. For example, an effective amount is an amount that reduces tumor growth by at least about 10%, at least about 20%, at least about 25%, at least about 30%, at least about 35%, at least about 40%, at least about 50%, at least about 60%, at least about 70%, at least about 80%, at least about 90% compared to tumor growth in the absence of the heme and at least one PAMP and/or DAMP therapy. Whether tumor growth decreases can be determined by measuring, e.g., actual tumor size, or any symptom associated with cancer such as weight, fatigue, fever, changes in bowel or bladder function, and the like. Treatment or a reduction in tumor growth can also extend survival and improve quality of life.

Cancers that can be treated in accordance with the methods herein include, but are not limited to, acute lymphoblastic leukemia, acute myeloid leukemia, adrenocortical carcinoma, anal cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, cervical cancer, chordoma, chronic lymphocytic leukemia, chronic myeloproliferative disorder, colon cancer, colorectal cancer, craniopharyngioma, cutaneous T cell lymphoma, endometrial cancer, ependymoblastoma, ependymoma, esophageal cancer, Ewing sarcoma, extracranial germ cell tumor, extragonadal germ cell tumor, extrahepatic bile duct cancer, eye cancer, gallbladder cancer, gastric cancer, gastroesophageal cancer, gastrointestinal cancer, germ cell tumor, gestational trophoblastic tumor, glioma (e.g., glioblastoma, astrocytoma, or oligodendrocytoma), hairy cell leukemia, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lung cancer, malignant teratoma, non-Hodgkin lymphoma, macroglobulinemia, osteosarcoma, medulloblastoma, melanoma, merkel cell carcinoma, mesothelioma, mouth cancer, mycosis fungiodes, myelodysplasia syndrome, multiple myeloma, nasal cavity and paranasal sinus cancer, nasopharyngeal cancer, non-small cell lung cancer, oral cancer, oropharyngeal cancer, osteosarcoma, ovarian cancer, ovarian epithelial cancer, pancreatic cancer, papillomatosis, parathyroid cancer, penile cancer, pharyngeal cancer, pituitary tumor, prostate cancer, rectal cancer, renal cell cancer, retinoblastoma, rhabdomyosarcoma, salivary gland cancer, sarcoma, skin cancer, small intestine cancer, soft tissue sarcoma, testicular cancer, throat cancer, thymic carcinoma, thyroid cancer, urethral cancer, uterine cancer, vaginal cancer, and Wilms tumor.

The present invention also provides methods for the manufacture of a medicament for treating a cancer or other proliferative disease or clearing an infection and kits for carrying out such methods. The kits of the invention include heme and at least one PAMP and/or DAMP molecule. The kits may provide a single dose or multiple doses of heme and at least one PAMP and/or DAMP molecule, wherein the heme and at least one PAMP and/or DAMP molecule may be provided individually or in a co-formulation. The kit may take the form of a blister package; a lidded blister, a blister card or packet; a clamshell; an intravenous (IV) package, IV packette or IV container, a tray or a shrink wrap comprising the heme and at least one PAMP and/or DAMP molecule and instructions for use of the composition in methods of treatment.

In so far as the data provided herein provides markers for heme- and PAMP/DAMP-mediated production or activation of NLRP12 or NLRC5 and inflammatory cell death, the present invention also provides a method of identifying an agent that inhibits NLRP12-dependent or NLRC5-dependent inflammasome, PANoptosome and inflammatory signaling activation, and inflammatory cell death. This screening method of the invention includes the steps of contacting a sample of cells with heme and at least one PAMP or DAMP molecule to activate NLRP12 or NLRC5 in the cells, and contacting the sample of cells with at least one test agent, wherein a decrease in the production or activation of NLRP12 or NLRC5 in the presence of the test agent as compared to production or activation of NLRP12 or NLRC5 in the absence of the test agent is indicative of an agent that inhibits NLRP12-dependent or NLRC5-dependent inflammasome and inflammatory signaling activation and inflammatory cell death. Accordingly, this method finds use in identifying NLRP12 and/or NLRC5 inhibitors that inhibit the expression or activity of NLRP12 and/or NLRC5 as well as inhibitors of upstream regulatory molecules of NLRP12 or NLRC5 including, e.g., TLRs, ROS, nicotinamide pathway components, and/or IRFs.

As used herein, the terms “sample” and “biological sample” refer to any sample suitable for the methods provided by the present invention. In one aspect, the biological sample of the present invention is a tissue sample, e.g., a biopsy specimen such as samples from needle biopsy (i.e., biopsy sample). In other embodiments, the biological sample of the present invention is a sample of bodily fluid, e.g., serum, plasma, sputum, lung aspirate, urine, and ejaculate.

In some aspects, activation of NLRP12 and/or NLRC5 is determined by measuring mRNA and/or protein levels or amounts of NLRP12 or NLRC5 (e.g., changes in NLRP12 and/or NLRC5 mRNA and/or protein levels); the cleavage of one or more of gasdermin D (GSDMD), GSDME, caspase-1, caspase-8, caspase-3, caspase-7; phosphorylation of mixed-lineage kinase domain-like (MLKL) protein; cell death; or release of IL-1β or IL-18 by the cells of the sample. Methods for assessing cleavage of GSDMD, GSDME, caspase-1, caspase-8, caspase-3, caspase-7; phosphorylation of MLKL protein; cell death; and release of IL-1β or IL-18 are described in the Examples herein.

As used herein, the term “test agent” or “candidate agent” refers to an agent that is to be screened in the assay described herein. The agent can be virtually any chemical compound. It can exist as a single isolated compound or can be a member of a chemical (e.g., combinatorial) library. In one embodiment, the test agent is a small organic molecule. The term small organic molecule refers to any molecules of a size comparable to those organic molecules generally used in pharmaceuticals. The term excludes biological macromolecules (e.g., proteins, nucleic acids, etc.). In certain embodiments, small organic molecules range in size up to about 5000 Da, up to 2000 Da, or up to about 1000 Da. Test agents of this invention may also be a peptide, antisense oligonucleotide, RNA hairpin, guide DNA, antibody or an antibody fragment.

In some aspects, the inhibitor identified in the screening assay of this invention is an inhibitory nucleic acid that inhibits the expression of NLRP12 or NLRC5. As used herein, an “inhibitory nucleic acid” means an RNA, DNA, or a combination thereof that interferes or interrupts the translation of mRNA. Inhibitory nucleic acids can be single or double stranded. The terms “short-inhibitory RNA” and “siRNA” interchangeably refer to short double-stranded RNA oligonucleotides that mediate RNA interference (also referred to as “RNA-mediated interference” or “RNAi”). The terms “small hairpin RNA” and “shRNA” interchangeably refer to an artificial RNA molecule with a tight hairpin turn that can be used to silence target gene expression via RNAi. RNAi is a highly conserved gene silencing event functioning through targeted destruction of individual mRNA by a homologous double-stranded small interfering RNA (siRNA) (Fire et al. (1998) Nature 391:806-811). Mechanisms for RNAi are reviewed, for example, in Bayne & Allshire (2005) Trends in Genetics 21:370-73; Morris (2005) Cell. Mol. Life Sci. 62:3057-66; Filipowicz et al. (2005) Current Opin. Struct. Biol. 15:331-41. In various aspects, the inhibitory nucleic acid is selected from the group of siRNA, shRNA, gRNA, oligonucleotides, antisense RNA or ribozymes that inhibit NLRP12 or NLRC5 synthesis. Suitable siRNA for decreasing the expression of NLRP12 or NLRC5 are known in the art and available from commercial sources such as Thermo Fisher. Similarly, a suitable shRNA plasmid to inhibit NLRP12 or NLRC5 expression by RNA interference is available from Abbexa Ltd. When using an gRNA for said inhibition of NLRP12 or NLRC5 expression, any suitable CRISPR system may be used including, but not limited to, Cas9. Exemplary gRNA/crRNA for genome editing with wild-type SpCas9 vector or Cas9 protein are available from GenScript.

The nucleotides of the inhibitory nucleic acid can be chemically modified, natural or artificial. With regard to antisense, siRNA or ribozyme oligonucleotides, phosphorothioate oligonucleotides can be used. Modifications of the phosphodiester linkage as well as of the heterocycle or the sugar may provide an increase in efficiency. Phophorothioate is used to modify the phosphodiester linkage. An N3′-P5′ phosphoramidate linkage has been described as stabilizing oligonucleotides to nucleases and increasing the binding to RNA. Peptide nucleic acid (PNA) linkage is a complete replacement of the ribose and phosphodiester backbone and is stable to nucleases, increases the binding affinity to RNA, and does not allow cleavage by RNAse H. Its basic structure is also amenable to modifications that may allow its optimization as an antisense component. With respect to modifications of the heterocycle, certain heterocycle modifications have proven to augment antisense effects without interfering with RNAse H activity. An example of such modification is C-5 thiazole modification. Finally, modification of the sugar may also be considered. 2′-O-propyl and 2′-methoxyethoxy ribose modifications stabilize oligonucleotides to nucleases in cell culture and in vivo.

Inhibitory oligonucleotides can be delivered to a cell by direct transfection or transfection and expression via an expression vector. Appropriate expression vectors include mammalian expression vectors and viral vectors, into which has been cloned an inhibitory oligonucleotide with the appropriate regulatory sequences including a promoter to result in expression of the antisense RNA in a host cell. Suitable promoters can be constitutive or development-specific promoters. Transfection delivery can be achieved by liposomal transfection reagents, known in the art (e.g., XTREME transfection reagent, Roche, Alameda, CA; LIPOFECTAMINE formulations, Invitrogen, Carlsbad, CA). Delivery mediated by cationic liposomes, nanoparticles, by retroviral vectors and direct delivery are efficient. Another possible delivery mode is targeting using antibody to cell surface markers for the target cells.

NLRP12 and/or NLRC5 inhibitors of this invention, e.g., those identified in the screening assay herein, find use in blocking NLRP12-mediated and/or NLRC5-mediated inflammasome, PANoptosome and inflammatory signaling activation and ameliorating or treating inflammation during hemolytic disease, infectious disease, inflammatory syndrome and cancer. Accordingly, in one aspect, the invention provides a method of treating or ameliorating NLRP12-mediated and/or NLRC5-mediated inflammation associated with a hemolytic disease, infectious disease or inflammatory syndrome in a subject in need thereof by administering to the subject an effective amount of an inhibitor of NLRP12 and/or NLRC5 activation or production. In some aspects, the inhibitor of NLRP12 or NLRC5 activation or production may directly inhibit the expression or activity of NLRP12 or NLRC5. For example, the inhibitor of NLRP12 or NLRC5 activation or production may be a small molecule, peptide, antisense oligonucleotide, guide RNA, shRNA, antibody or an antibody fragment that interacts with NLRP12 or NLRC5 or nucleic acids encoding NLRP12 or NLRC5 and inhibits NLRP12 or NLRC5 expression or activity. In other aspects, the inhibitor of NLRP12 or NLRC5 activation or production may target an upstream regulatory molecule of NLRP12 or NLRC5, e.g., a Toll-like receptor (TLR), reactive oxygen species (ROS), and/or interferon regulatory factor (IRF) thereby inhibiting the production or activation of NLRP12 or NLRC5. An inhibitor targeting an upstream regulatory molecule of NLRP12 or NLRC5 may be a small molecule, peptide, antisense oligonucleotide, guide RNA, shRNA, antibody or an antibody fragment.

The terms “treat,” “treating” or “treatment” refer to an approach for obtaining beneficial or desired clinical results. For purposes of this invention, beneficial or desired clinical results include, but are not limited to, treatment of NLRP12-mediated and/or NLRC5-mediated inflammation. As used herein, the term “ameliorate” means that the clinical signs and/or the symptoms associated with NLRP12-mediated and/or NLRC5-mediated inflammation are lessened. The signs or symptoms to be monitored will be characteristic of a particular disease or disorder and will be well known to the skilled clinician, as will the methods for monitoring the signs and conditions thereof.

As indicated, the NLRP12-mediated and/or NLRC5-mediated inflammation may be associated with hemolytic diseases including, but not limited to, beta thalassemia, hemolytic anemia, and sickle cell disease (SCD); infectious diseases including, but not limited to, SARS-CoV-2, malaria, influenza, and pneumonia; and inflammatory syndromes including, but not limited to, neurological inflammation (e.g., schizophrenia, ALS, Alzheimer's, dementia) or systemic inflammatory response syndrome (SIRS).

Generally, compositions of this invention, e.g., heme and at least one PAMP and/or DAMP molecule or an NLRP12 and/or NLRC5 inhibitor, are formulated so as to be suitable for administration to the subject, which can be any vertebrate subject, including a mammalian subject (e.g., a human subject). Such formulated compositions are useful as medicaments for treating a subject suffering from any of the above-mentioned diseases.

Preferably, the compositions of this invention include isolated molecules. An isolated molecule is a molecule that is substantially pure and is free of other substances with which it is ordinarily found in nature or in vivo systems to an extent practical and appropriate for its intended use. In particular, the molecular species are sufficiently pure and are sufficiently free from other biological constituents of host cells so as to be useful in, for example, producing pharmaceutical preparations or sequencing if the molecular species is a nucleic acid or peptide. Because an isolated molecular species of the invention may be admixed with a pharmaceutically acceptable carrier in a pharmaceutical preparation, the molecular species may include only a small percentage by weight of the preparation. The molecular species is nonetheless substantially pure in that it has been substantially separated from the substances with which it may be associated in living systems.

Compositions used in the methods of this invention can be provided in a pharmaceutical composition including the composition in admixture with at least one pharmaceutically acceptable carrier, excipient, vehicle, diluent, or preservative. The pharmaceutically acceptable carrier preferably is non-pyrogenic and may include, but is not limited to, saline, buffered saline, dextrose, and water. A variety of aqueous carriers may be employed, e.g., 0.4% saline, 0.3% glycine, and the like. These solutions are sterile and generally free of particulate matter. These solutions may be sterilized by conventional, well-known sterilization techniques (e.g., filtration).

The pharmaceutical compositions may contain pharmaceutically acceptable auxiliary substances as required. Acceptable auxiliary substances preferably are nontoxic to recipients at the dosages and concentrations employed. Auxiliary substances may be used to maintain or preserve, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCL, citrates, phosphates, or other organic acids), bulking agents (such as mannitol or glycine), chelating agents (such as ethylenediamine tetra acetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring agents, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics, PEG, sorbitan esters, polysorbates such as polysorbate 20 or polysorbate 80, tromethamine, lecithin, or cholesterol), stability enhancing agents (such as sucrose or sorbitol), tonicity enhancing agents (such as alkali metal halides), delivery vehicles, diluents, excipients and/or pharmaceutical adjuvants. See Remington's Pharmaceutical Sciences (18th Ed., A. R. Gennaro, ed., Mack Publishing Company 1990).

Yet another preparation can involve the formulation of a composition disclosed herein in an injectable microsphere, bio-erodible particle, polymeric compound (such as polylactic acid or polyglycolic acid), bead, or liposome, that provides for the controlled or sustained release of the product which may then be delivered via a depot injection. Other suitable means for the introduction of the desired inhibitor include implantable drug delivery devices.

A composition to be used for in vivo administration typically must be sterile. This may be accomplished by filtration through sterile filtration membranes. Where the composition is lyophilized, sterilization using this method may be conducted either prior to, or following, lyophilization and reconstitution.

The concentration of the active components of the composition can vary widely, i.e., from less than about 0.5%, usually at or at least about 1% to as much as 15 or 20% by weight and will be selected primarily based on fluid volumes, viscosities, etc., according to the particular mode of administration selected.

Compositions of the invention can be administered by any number of routes as described herein including, but not limited to, intravenous, intramuscular, intra-arterial, intramedullary, intrathecal, intraventricular, intracerebroventricular, intrathecal-lumbar, intracisternal, transdermal, subcutaneous, intraperitoneal, intranasal, intratumoral, parenteral, topical, sublingual, or rectal means.

The following non-limiting examples are provided to further illustrate the present invention.

### Example 1: Materials and Methods

Mice. Casp1^{-/-} (Man et al. (2016) Cell 167:382-396), Casp3^{-/-} (Zheng et al. (2000) Nat. Med. 6:1241-1247), Casp7^{-/-} (Lakhani et al. (2006) Science 311:847-851), Ripk3^{-/-} (Newton et al. (2004) Mol. Cell. Biol. 24:1464-1469), Nlrp1b^{-/-} (Kovarova et al. (2012) J. Immunol. 189:2006-2016), Nlrp3^{-/-} (Kanneganti et al. (2006) Nature 440:233-236), Nlrp6^{-/-} (Chen et al. (2011) J. Immunol. 186:7187-7194), Nlrp12^{-/-} (Zaki et al. (2011) Cancer Cell 20:649-660), and Nlrc5^{-/-} (Kumar et al. (2011) J. Immunol. 186:994-10000) mice have been previously described. Casp1^{-/-}Casp8^{-/-}Ripk3^{-/-} mice were bred by crossing Ripk3^{-/-}Casp8^{-/-} (Oberst et al. (2011) Nature 471:363-367) with Casp1^{-/-} mice. Nlrp12^{-/-}Nlrc5^{-/-} mice were bred by crossing Nlrp12^{-/-} (Zaki et al. (2011 Cancer Cell 20:649-660) with Nlrc5^{-/-} mice (Kumar et al. (2011) J. Immunol. 186:994-10000). All mice were generated on or extensively backcrossed to the C57/BL6 background. Mice were bred at the Animal Resources Center at St. Jude Children's Research Hospital and maintained under specific pathogen-free conditions. Male age- and sex-matched 8- to 10-week-old mice were used in this study. Mice were maintained with a 12-hour light/dark cycle and were fed standard chow. Animal studies were conducted under protocols approved by the St. Jude Children's Research Hospital committee on the Use and Care of Animals. WT and Nlrp12^{-/-} animals or WT and Nlrc5^{-/-} animals were co-housed for 2 weeks before performing in vivo experiments.

Bone Marrow-Derived Macrophages (BMDMs). Primary mouse BMDMs from wild-type and indicated mutant mice were grown for 6 days in Iscove's Modified Dulbecco's Medium (IMDM; Thermo Fisher Scientific) supplemented with 10% Fetal Bovine Serum (Biowest), 30% L929-conditioned media, 1% non-essential amino acids (Thermo Fisher Scientific) and 1% penicillin and streptomycin (Thermo Fisher Scientific). BMDMs at a density of 1×10⁶ cells/well in 12-well plates or 5×10⁵ cells/well in 24-well plates were seeded into growth media overnight before use.

Cell Stimulation. BMDMs were stimulated with the following PAMPs, DAMPs and inhibitors alone or in combinations where indicated: 50 μM Hemin (heme; Sigma-Aldrich), 0.75 μg/mL Pam3CSK4 (Pam3; InvivoGen), 15 ng/mL ultrapure lipopolysaccharide (LPS) from E. coli 0111:B4 (InvivoGen), 500 ng/ml R848 (InvivoGen), 100 ng/mL TNF-α (Peprotech), 1 μg/mL poly(I:C) (Invivogen), 200 ng/mL monosodium urate crystals (Invivogen), 200 ng/mL recombinant mouse S100A8/S100A9 heterodimer (bio-techne), 200 ng/mL recombinant mouse HMGB1 (Abcam), 25 μM Z-VAD(OMe)-FMK (zVAD; Cayman Chemical) and 45 μM Necrostatin 2 racemate (Nec-1; Selleckchem).

Real-Time Imaging for Cell Death. The kinetics of cell death were monitored using the IncuCyte S3 or SX5 (Sartorius) live-cell analysis system. BMDMs (5×10⁵ cells/well) were seeded in 24-well tissue culture plates and were treated with the indicated stimuli. Cell death was measured by propidium iodide (PI; Life Technologies) incorporation following the manufacturer's protocol. The plate was scanned for the indicated time durations where fluorescent and phase-contrast images were acquired in real-time every 1 hour. PI-positive dead cells are marked with a red mask and were quantified using the software package supplied with the IncuCyte imager.

Immunoblot Analysis. After appropriate treatments, cells were lysed along with culture supernatants in caspase lysis buffer (containing 10% NP-40, 25 mM DTT and 1× protease and phosphatase inhibitors) and SDS sample loading buffer (with 2-mercaptoethanol) for probing caspase activation. For immunoblot assessment of signaling activation, culture supernatants were removed, cells were washed once with 1×DPBS and lysed in RIPA buffer and SDS sample loading buffer. Proteins were resolved on 8-12% polyacrylamide gels and transferred onto PVDF membranes (Millipore) using the Trans-BLOT® TURBO™ system. After blocking non-specific binding with 5% skim milk, membranes were incubated overnight with the following primary antibodies against: caspase-1 (AdipoGen), caspase-3 (Cell Signaling Technology [CST]), cleaved caspase-3 (CST), caspase-7 (CST), cleaved caspase-7 (CST), caspase-8 (AdipoGen), cleaved caspase-8 (CST), pMLKL (CST), tMLKL (Abgent), GSDMD (Abcam), GSDME (Abcam), HO-1 (CST) and β-actin (Santa Cruz). Membranes were then washed and probed with the appropriate horseradish peroxidase (HRP)-conjugated secondary antibodies (anti-mouse and anti-rabbit; Jackson ImmunoResearch Laboratories). Immunoblot images were acquired on an Amersham Imager using IMMOBILON® Forte Western HRP Substrate (Millipore).

Cytokine Measurement. In vitro cytokines were detected in the supernatant by using multiplex ELISA (Millipore) and IL-18 ELISA (Invitrogen), according to the manufacturer's instructions.

Microarray and RNA-seq Analysis. Seven datasets deposited in GEO (accession IDs: GSE34404 (Idaghdour et al. (2012) Proc. Natl. Acad. Sci. USA 109:16786-16793), GSE136046 (Brito et al. (2022) J. Infect. Dis. 225:1274-1283), GSE102881 (Lagresle-Peyrou et al. (2018) Haematologica 103:778-786), GSE168532 (Lagresle-Peyrou et al. (2018) Haematologica 103:778-786; Liu et al. (2021) Blood 138:1162-1171), GSE58287 (Connor et al. (2015) J. Virol. 89:9865-9874), GSE40012 (Parnell et al. (2012) Crit. Care 16:R157) and GSE171110 (Levy et al. (2021) iScience 24:102711)) were used to estimate the role of NLRPs in datasets relevant for hemolytic and pandemic diseases. GSE34404 (Idaghdour et al. (2012) Proc. Natl. Acad. Sci. USA 109:16786-16793) compared whole blood RNA-seq profiles of 155 West-African children, including 94 cases undergoing symptomatic phases of Plasmodium falciparum infection and 61 age-matched controls. GSE136046 (Brito et al. (2022) J. Infect. Dis. 225:1274-1283) included expression profiles of affinity purified CD71⁺ cells from three patients with Plasmodium vivax at day 1 (diagnosis visit) and day. 42 after curative drug treatment (convalescence visit). From GSE102881 (Lagresle-Peyrou et al. (2018) Haematologica 103:778-786), RNA-seq profiles were obtained from CD34⁺ hematopoietic stem/progenitor cells (HSPCs) collected from the bone marrow of two healthy donors and two patients with sickle cell disease (SCD). GSE168532 (Lagresle-Peyrou et al. (2018) Haematologica 103:778-786; Liu et al. (2021) Blood 138:1162-1171) included transcriptomic profiles of classical monocytes from the peripheral blood of six healthy controls and 13 patients with SCD. GSE58287 (Connor et al. (2015) J. Virol. 89:9865-9874) included temporal transcriptomic profiles from 30 peripheral blood mononuclear cells from 15 cynomolgus macaques infected with Marburg virus. The dataset included transcriptomic profiles of 15 macaques at day 0 and three macaques post-infection at days 1, 3, 5, 7 and 9.

GSE40012 (Parnell et al. (2012) Crit. Care 16:R157) included whole blood samples of patients in critical care for up to 5 days and assayed on Illumina HT-12 gene expression bead arrays. The dataset included patients with influenza A infection (n=11), bacterial pneumonia (n=16) and systemic inflammatory response (n=13) along with healthy control samples (n=36). To compare a severe disease phenotype with healthy controls while maintaining maximum patient samples per phenotype, patient samples at day 4 for influenza (n=11), pneumonia (n=10) and systemic inflammatory response (n=6) were considered. GSE171110 (Parnell et al. (2012) Crit. Care 16:R157) included whole blood transcriptomics for 44 patients with severe COVID-19 and 10 healthy controls for comparison. Additionally, a cohort of 18 patients (Sequence Read Archive: PRJNA680886; Neogi et al. (2022) Elife 11) infected with Crimean-Congo Hemorrhagic Fever Viruses (CCHFV) was obtained, and blood transcriptomics were available for 12 patients with severe CCHFV. This allowed for a differential expression analysis for patients infected with CCHFV between the time of infection and approximately one-year post-recovery (convalescent group).

For each of these datasets, quality control steps were performed, including normalized quantiles using the ‘normalize.quantiles’ function from preprocessCore v1.58.0 package, when the counts were not normalized, followed by log 2 transformation for downstream differential expression analysis. Differential expression analysis was performed using the limma v3.52.1 (Love et al. (2014) Genome Biol. 15:550) package in R v4.1.1. The Benjamini-Hochberg (Benjamini & Hochberg (1995) J. R. Stat. Soc. Series B Stat. Methodol. 57:289-300) adjusted P value <0.05 for GSE34404, GSE168532, GSE40012 and GSE171110 was used to determine the set of differentially expressed genes. However, owing to the small sample sizes for GSE136046 (three cases and three controls), GSE102881 (two cases and two controls) and GSE58287 (three cases for each of day 1, 3, 5, 7 and 9), P value adjustments were not made for these three datasets. A P value <0.05 was used to estimate the set of differentially expressed genes for GSE136046 and GSE102881. The cytosolic sensors, in particular the NLRPs, were assessed to determine which were overexpressed across the disease-relevant datasets. The NLRPs were sorted based on their average fold-change across these datasets and were visualized using Heatmap from the Complex Heatmap v2.8.0 (Gu et al. (2016) Bioinformatics 32:2847-2849) package.

Single Cell Analysis. Single cell transcriptomics data were obtained from GSE133181 (Hua et al. (2019) Blood 134:2111-2115). This dataset was composed of single cells obtained from the bone marrow CD34⁺ cells of 4 normal patients (BM), 3 patients with thalassemia major (BT) and 5 patients with SCD analyzed through the 10× chromium platform. The original dataset included 32,389 cells of BM, 9,862 cells of BT and 16,266 cells of SCD, along with expression profiles for 33,694 genes, and was analyzed using Seurat v4.1.1 package in R v4.1.1. Quality control steps were performed as suggested previously (Hua et al. (2019) Blood 134:2111-2115). Cells were excluded if the number of genes detected was below 500 or the percentage of mitochondrial genes was above 5%. Log-normalization was performed using the ‘NormalizeData’ function on a scale factor as 10000. All variable genes passing the ‘vst’ filter were included for further analysis. Biological variation within the same group was removed by the ‘ScaleData’ function. Principal component analysis was performed using the ‘RunPCA’ function. The top 20 principal components (PCs) were used in downstream analysis. Louvain graph-based clustering and t-SNE based dimensionality reduction were also performed to obtain low-dimensional representations and visualize the dataset.

To determine the cell type annotation, a set of known markers and transcription factors for bone marrow cell types were used as described previously (Hua et al. (2019) Blood 134:2111-2115). Each cell was annotated using the ‘AUCell_buildRankings’, ‘AUCell_calcAUC’ and ‘AUCell_exploreThresholds’ functions (in order) from the AUCell v1.18.0 package.

The final dataset was composed of 24,864 cells of BM, 6,159 cells of BT and 8,018 cells of SCD and were distributed among four cell types including: Lymphoid (Lym_P), Myeloid (G/M_P), Erythroid (M/E_P) and Multipotential Hematopoietic Stem (HSPC1) progenitor cells. A nonparametric Mann-Whitney-Wilcoxon test (Hettmansperger & McKean (2010) Robust Nonparametric Statistical Methods (2nd ed.) CRC Press) was used to compare average NLRP12 expression between BT vs BM and SCD vs BM cells bifurcated by the various cell types.

Confocal Microscopy. BMDMs were seeded onto poly-D-lysine coated coverslips. After appropriate treatments, cells were washed with DPBS, fixed with 4% paraformaldehyde and permeabilized with 0.5% TRITON X-100. After blocking non-specific bindings with 10% normal goat serum, the cells were incubated overnight at 4° C. with primary antibody against ASC (1:100, Millipore). After three washes with PBS-T (0.05% polysorbate 20 in PBS), the coverslips were incubated with ALEXA FLUOR®488 dye-conjugated secondary antibody against mouse IgG (1:1000, Invitrogen) for 2 hours at room temperature. Cells were counterstained with DAPI (4′,6-Diamidino-2-Phenylindole, Biotium) to visualize nuclei, and images were acquired using Marianas spinning disc confocal system (Intelligent Imaging Innovations) composed of an inverted AxioObserver Z.1 microscope (Carl Zeiss), CSU-W1 with SoRa (Yokogawa), Prime95B sCMOS camera (Photometrics), 405 nm, 473 nm, 561 nm, 647 nm solid state laser lines (Coherent) and a 1.4 NA 60× oil objective. Images were acquired using Slidebook software.

RT-PCR Analysis. Total RNA was extracted at indicated time points using TRIZOL® RNA extraction reagent (Thermo Fisher Scientific). cDNA was synthesized with 1 μg of extracted RNA using the High-Capacity cDNA Reverse Transcription Kit (Applied Biosystems). Real-time quantitative PCR was performed with SYBR® Green (Applied Biosystems) as the fluorescent reporter on an Applied Biosystems 7500 real-time PCR instrument. The mouse primer sequences used are as follows: mNlrp12—Forward primer: AAGACCGCAATGCACGATTAG (SEQ ID NO:1); Reverse primer: TGGAGCGTTCCCACTCTACA (SEQ ID NO:2); mActin—Forward primer: GGCTGTATTCCCCTCCATCG (SEQ ID NO:3); Reverse primer: CCAGTTGGTAACAATGCCATGT (SEQ ID NO:4); mNlrc5—Forward primer: GTGCCAAACGTCCTTTTCAGA (SEQ ID NO:5); Reverse primer: AGTGAGGAGTAAGCCATGCTC (SEQ ID NO:6).

Hemin Preparation. Hemin (heme; ferriprotoporphyrin IX chloride, Sigma-Aldrich) 100 mM stock was prepared by dissolving with filter sterilized 0.1 M NaOH and neutralizing (to pH 7.2) with 1 M HCl, as previously described (Rossi et al. (2018) Biochem. Biophys. Res. Commun. 503:2820-2825). Prepared stocks were aliquoted and stored at −80° C. until used.

In Vivo Injection. LPS, 1 μg/g body weight (Sigma), and phenylhydrazine, 0.125 mg/g body weight (Sigma), were used for in vivo experiments. Briefly, LPS was dissolved in sterile DPBS, aliquoted and stored in −80° C. until used. Phenylhydrazine was weighed and dissolved in sterile DPBS; then the pH adjusted to 7.4 using 2 M NaOH, and phenylhydrazine was filtered through a 0.22 μm syringe filter (Millipore). Age- and sex-matched, 6- to 8-week-old cohoused mice of the indicated genotypes were injected intraperitoneally with 1 μg/g body weight LPS alone, 0.125 mg/g body weight phenylhydrazine alone or LPS with phenylhydrazine. For LPS-phenylhydrazine combination injections, LPS was added to sterile, filtered phenylhydrazine and mixed before injection. For Plasmodium berghei ANKA infection, 6- to 8-week-old cohoused mice of the indicated genotypes were injected intraperitoneally with 10⁵ infected RBCs (iRBCs) per mouse. Infected animals were monitored for survival every day.

Clinical Chemistry Analysis. Serum heme was detected using a heme assay kit (Sigma). BUN, creatinine, AST and iron were detected using ABX Pentra 400 Reagents (HORIBA) according to the manufacturer's instructions.

Statistical Analysis. GraphPad Prism 8.0 software was used for data analysis. Data are shown as mean±SEM. Statistical significance was determined by t tests (two-tailed) for two groups or one- or two-way ANOVA (with Dunnett's or Tukey's multiple comparisons tests) for three or more groups. P value <0.05 was considered statistically significant.

### Example 2: Identification of Unique PAMP and DAMP Combinations that Induce Inflammatory Cell Death

Innate immunity is activated in response to pathogens, PAMPs or DAMPs. Enhanced immune activation can lead to excess cell death, tissue destruction and organ damage, as well as the release of DAMPs. PRRs have classically been studied for their ability to sense a specific PAMP or DAMP. However, combined activation of PRRs by multiple PAMPs and DAMPs together has not been well characterized. It was hypothesized that the presence of multiple stimuli would mimic infection and drive inflammatory cell death.

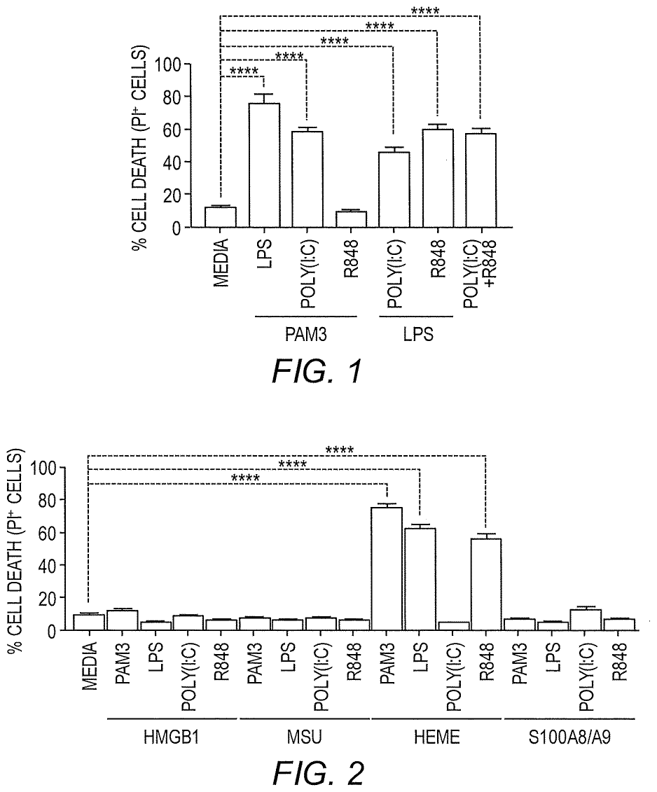

To study the involvement of multiple PAMPs in cell death and search for combinations that could mimic infection, lipopolysaccharides (LPS) and Pam3CSK4 (Pam3), which mimic bacterial infection, and polyinosinic:polycytidylic acid (poly(I:C)) and Resiquimod (R848), which mimic viral infection, were tested. To test whether individual PAMPs alone could induce cell death, bone marrow-derived macrophages (BMDMs) were treated with Pam3, poly(I:C), LPS or R848. Minimal cell death occurred with these triggers. However, when PAMPs were combined, as would be expected to occur in natural infection, robust cell death was observed in many combinations (FIG. 1).

Because tissue damage and DAMP release often also occur during infections and inflammatory conditions, the involvement of DAMPs in cell death was also determined. To assess this, DAMPs associated with infections and inflammatory diseases, e.g., high-mobility group box 1 protein (HMGB1), monosodium urate (MSU), S100A8/A9 and heme were used. Similar to what was observed with PAMPs, it was found that individual DAMPs did not induce cell death in BMDMs. Similarly, combinations of DAMPs did not induce cell death.

It was subsequently determined whether combinations of PAMPs with DAMPs could induce cell death. Notably, robust cell death was observed specifically when heme was combined with Pam3, LPS or R848, but not in other combinations tested, including heme with poly(I:C) (FIG. 2). Together, these results indicate that signaling driven by specific PAMP combinations and the combination of heme with PAMPs could induce robust cell death.

While heme is known to induce cell death (Fortes et al. (2012) Blood 119:2368-2375), the cytosolic sensors and molecular mechanisms involved remain unclear. Therefore, the biochemical features of the cell death induced by the combination of heme and PAMPs were subsequently elucidated. Caspase-1 cleavage was initially observed, indicating the involvement of inflammasome activation. Downstream of inflammasome activation, gasdermin D (GSDMD) can be processed to release its pore-forming N-terminus to execute cell death (He et al. (2015) Cell Res. 25:1285-1298; Kayagaki et al. (2015) Nature 526:666-671; Shi et al. (2015) Nature 526:660-665). Consistent with the caspase-1 activation, cleavage of GSDMD to this pore-forming P30 fragment was observed, though at low levels. Another member of the gasdermin family, gasdermin E (GSDME), has been shown to induce cell death under specific conditions (Wang et al. (2017) Nature 547:99-103), and GSDME cleavage was also observed herein. In addition, cleavage of apoptotic caspases, caspase-8, -3 and -7, was observed in BMDMs treated with heme plus PAMPs. Previous studies have shown that activation of caspase-3 and -7 can inactivate GSDMD by processing it to produce a P20 fragment (Chen et al. (2019) The EMBO J. 38:e101638; Taabazuing et al. (2017) Cell. Chem. Biol. 24:507-514; Wang et al. (2021) J. Immunol. 207:2411-2416), which was also observed herein. Furthermore, phosphorylation of MLKL was also detected. Collectively, these data indicate that synergistic signaling from heme and PAMPs induces inflammatory cell death characterized by activation of caspase-1, GSDMD, GSDME, caspases-8, -3, -7 and MLKL, hallmarks of PANoptosis.

### Example 3: NLRP12 Regulates Inflammatory Cell Death Induced by Heme Plus PAMPs

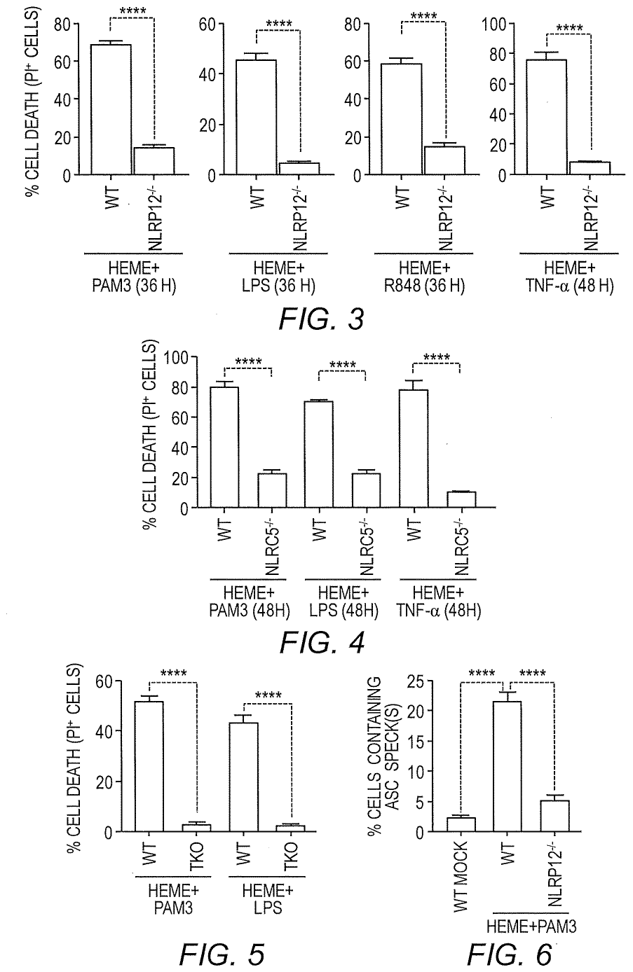

Cytosolic sensors, and particularly some NLR proteins, are implicated in the induction of cell death and disease (Kanneganti (2020) Immunol. Rev. 297:5-12; Harton et al. (2002) J. Immunol. 169:4088-4093; Inohara & Nunez (2003) Nat. Rev. Immunol. 3:371-382). However, the roles of many NLRs in innate immunity and cell death remain unclear. To understand which NLRPs may be important in hemolytic diseases, publicly available datasets for patients with malaria or sickle cell disease (SCD) (Idaghdour et al. (2012) Proc. Natl. Acad. Sci. USA 109:16786-16793; Brito et al. (2022) J. Infect. Dis. 225:1274-1283; Lagresle-Peyrou et al. (2018) Haematologica 103:778-786) were re-analyzed. While earlier studies suggested that heme can induce NLRP3 inflammasome activation and IL-1p secretion (Li et al. (2014) Cell. Biochem. Biophys. 69:495-502; Dutra et al. (2014) Proc. Natl. Acad. Sci. USA 111:E4110-4118), it was observed that NLRP12, NLRP3 and NLRP6 were significantly upregulated in malaria or SCD, whereas NLRP2 and NLRP1 were not significantly upregulated. To confirm whether these upregulated NLRPs played a role in inflammatory cell death during hemolytic diseases, cell death in NLRP12-, NLRP3-, NLRP6- and NLRP1b-deficient BMDMs stimulated with heme plus PAMPs was evaluated. It was observed that Nlrp12^{-/-} BMDMs were significantly protected from cell death (FIG. 3), while there was moderate protection in Nlrp3^{-/-} BMDMs, and no significant cell death protection in Nlrp6^{-/-} and Nlrp1b^{-/-} BMDMs. To further confirm that NLRP3 was not involved in inducing the cell death, the NLRP3 inhibitor MCC950 (Coll et al. (2015) Nat. Med. 21:248-255) was used. Treatment with MCC950 prevented canonical NLRP3-mediated cell death in response to LPS plus ATP, but did not prevent cell death in heme plus Pam3-treated BMDMs. To further confirm the role of NLRP12 in driving cell death in response to heme plus PAMPs, WT and Nlrp12^{-/-} BMDMs were treated with heme and R848. Consistent with heme and Pam3 or LPS stimulations, it was found that Nlrp12^{-/-} BMDMs were significantly protected from cell death during heme and R848 treatment (FIG. 3). To determine whether NLRP12 was also involved in PAMP-PAMP combination-induced cell death, WT and Nlrp12^{-/-} BMDMs were stimulated with poly(I:C) and Pam3 or R848. However, there was no significant cell death protection observed in Nlrp12^{-/-} BMDMs compared to WT BMDMs, indicating that NLRP12 is not required to drive cell death in PAMP-PAMP combinations. Together, these data indicate that NLRP12 is specifically required to drive inflammatory cell death in response to heme plus PAMPs.

Previous studies have also shown that cell death, characterized by necroptotic and apoptotic activation, can be induced by heme plus TNF-α (Fortes et al. (2012) Blood 119:2368-2375; Seixas et al. (2009) Proc. Natl. Acad. Sci. USA 106:15837-15842). Therefore, it was determined whether NLRP12 was also important for the cell death induced by heme plus TNF-α. It was observed that Nlrp12^{-/-} BMDMs were significantly protected from cell death in response to heme plus TNF-α (FIG. 3). Excess circulating TNF-α is a feature of both infectious and inflammatory diseases (Bradley (2008) J. Pathol. 214:149-160), indicating that NLRP12 plays a role in heme-mediated cell death in both infections and autoinflammatory disease conditions.

### Example 4: NLRC5 Regulates Inflammatory Cell Death Induced by Heme Plus PAMPs

In addition to testing the role for NLRPs, it was also observed that NLRC5 played a role in response to heme plus PAMPs. It was observed that Nlrc5^{-/-} BMDMs were significantly protected from cell death in response to heme plus Pam3 and heme plus LPS stimulations (FIG. 4). To further confirm the role of NLRC5 in driving cell death in response to heme plus PAMPs, WT and Nlrc5^{-/-} BMDMs were treated with heme and R848. Consistent with heme and Pam3 or LPS stimulations, it was found that Nlrc5^{-/-} BMDMs were significantly protected from cell death during heme and R848 treatment. Additionally, it was observed that Nlrc5^{-/-} BMDMs were significantly protected from cell death in response to heme plus TNF-α (FIG. 4). Together, these data indicate that NLRC5 is specifically required to drive inflammatory cell death in response to heme plus PAMPs and cytokines.

### Example 5: NLRP12 and NLRC5 Regulate PANoptosis Induced by Heme Plus PAMPs

It was subsequently determined how the loss of NLRP12 or NLRC5 molecularly impacted cell death. In response to treatment with heme plus Pam3, decreased cleavage of GSDMD (P20 form), GSDME, caspases-8, -3, and -7 and reduced phosphorylation of MLKL in Nlrp12^{-/-} and Nlrc5^{-/-} BMDMs compared to WT BMDMs was observed, indicating that NLRP12 and NLRC5 regulate the activation of the inflammasome and inflammatory cell death molecules consistent with PANoptosis in response to heme plus PAMPs. It was hypothesized that the effect of NLRP12 and NLRC5 on cell death may be linked to heme oxygenase-1 (HO-1), which is upregulated in response to heme and is critical in the heme detoxifying process (Seixas et al. (2009) Proc. Natl. Acad. Sci. USA 106:15837-15842; Pamplona et al. (2007) Nat. Med. 13:703-710). Increased expression of HO-1 was observed in response to heme plus PAMPs; however, there was no difference in HO-1 expression between WT and Nlrp12^{-/-} BMDMs. These data indicated that the impact of NLRP12 on cell death is independent of HO-1 function. Together, these data indicate the NLRP12 and NLRC5 regulate PANoptosis, inflammatory cell death, in response to heme plus PAMPs.

### Example 6: NLRP12 and NLRC5 Drive Caspase-1/Caspase-8/RIPK3-Dependent Cell Death Induced by Heme Plus PAMPs

Because NLRP12-dependent and NLRC5-dependent activation of multiple cell death proteins, consistent with the activation of PANoptosis, was observed in response to heme plus PAMPs, including caspases-1, -3, -7 and MLKL, the contribution of each of these cell death proteins to the overall cell death phenotype was determined. To address this question, Casp1^{-/-}, Casp3^{-/-}, Casp7^{-/-} and Ripk3^{-/-} BMDMs were evaluated. No significant reduction in cell death was found between WT and Casp1^{-/-}, Casp3^{-/-}, Casp7^{-/-} or Ripk3^{-/-} BMDMs upon heme plus Pam3 or heme plus LPS stimulations, indicating that there could be redundancy among these cell death molecules in the execution of NLRP12^{-/-} and NLRC5-dependent cell death. To further assess the role of other cell death proteins in this phenotype, WT BMDMs were treated with the RIPK1 inhibitor, Nec-1, or the combination of Nec-1 with the pan-caspase inhibitor, z-VAD-FMK (zVAD). Nec-1 treatment partially reduced cell death in response to heme plus Pam3 compared to PBS control, and the addition of zVAD further reduced the cell death, indicating that RIPK1 and caspases are both important for cell death in response to heme plus Pam3.

Since significant cell death protection was observed in zVAD and Nec-1 treated BMDMs upon heme plus Pam3 stimulation, a genetic model was used to confirm these findings. In this analysis, Casp1^{-/-}Casp8^{-/-}Ripk3^{-/-} (referred to as triple knock-out [TKO]) BMDMs were used, where key components associated with PANoptosis were absent in these cells. Significant protection from cell death was observed in TKO BMDMs compared to WT BMDMs in response to heme plus Pam3 or heme plus LPS (FIG. 5). Consistent with cell death protection, reduced cleavage of GSDMD (P20 form), GSDME, caspases-3 and -7 was observed, as well as reduced phosphorylation of MLKL in TKO BMDMs compared to WT BMDMs upon heme plus Pam3 or heme plus LPS stimulation. Together, these results indicated that the NLRP12- and NLRC5-dependent inflammatory cell death in response to heme and PAMPs is driven by the caspase-1/caspase-8/RIPK3 axis.

### Example 7: NLRP12 and NLRC5 Drive Inflammasome Formation in Response to Heme Plus PAMPs

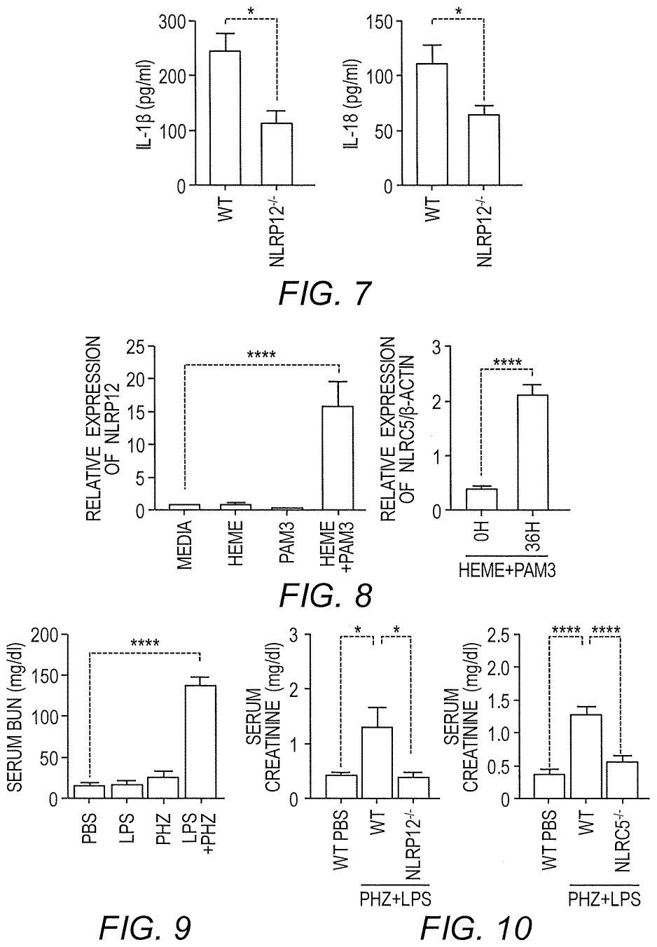

Caspase-1 activation is a hallmark of inflammasome activation. Additionally, NLRP12 has been suggested to act as an inflammasome sensor during Yersinia pestis or Plasmodium chabaudi infections (Vladimer et al. (2012) Immunity 37:96-107; Ataide et al. (2014) PLoS Pathog. 10:e1003885), and NLRC5 has been observed to act as a positive regulator of the NLRP3 inflammasome under certain conditions (Davis et al. (2011) J. Immunol. 186:1333-1337; Kumar et al. (2011) J. Immunol. 186:994-1000). However, NLRP12 also has inflammasome-independent functions to dampen NF-κB and ERK activation during inflammation and Salmonella infection (Allen et al. (2012) Immunity 36:742-754; Zaki et al. Proc. Natl. Acad. Sci. USA 111:385-390), and loss of NLRP12 in mice results in increased susceptibility to colon inflammation, colorectal tumor development and atypical neuroinflammation (Allen et al. (2012) Immunity 36:742-754; Allen et al. (2012) Immunity 36:742-754; Zaki et al. (2011) Cancer Cell 20:649-660; Lukens et al. (2015) Immunity 42:654-664); and NLRC5 may reduce NF-κB and IFN signaling pathways independent of the NLRP3 inflammasome (Cui et al. (2010) Cell 141:483-496). Therefore, the role of NLRP12 and NLRC5 in inflammasome formation and activity in response to heme plus PAMPs was evaluated. Initially, the formation of ASC specks, a hallmark of inflammasome formation, was examined. The results of this analysis indicated an increase in the formation of ASC specks in WT BMDMs upon heme plus Pam3 treatment when compared to untreated BMDMs (FIG. 6). Moreover, a significant reduction in ASC speck formation in Nlrp12^{-/-} BMDMs compared to WT BMDMs was observed (FIG. 6), indicating that NLRP12 is required for inflammasome assembly under these conditions. As a downstream readout of inflammasome activation, the release of IL-1β and IL-18, which are cleaved into their active forms by caspase-1, was assessed. Consistent with ASC speck formation, it was found that treatment with heme and Pam3 led to the release of IL-1β and IL-18 in WT BMDMs, while there was a significant reduction in cytokine release in Nlrp12^{-/-} BMDMs (FIG. 7). Additionally, loss of either NLPR12 or NLRC5 reduced caspase-1 activation in response to heme plus Pam3 stimulation. Together, these data indicate that NLRP12 and NLRC5 regulate inflammasome formation and activation in response to heme plus PAMPs.

### Example 8: NLRP12 and NLRC5 Form a Multiprotein Cell Death Complex Induced by Heme Plus PAMPs

A critical role for the inflammasome and the caspase-8 axis was observed in response to heme plus PAMP treatment. Inflammasomes and caspase-8 are integral components of PANoptosome complexes to induce cell death. Therefore, the potential for an interaction between NLRP12, NLRC5, and PANoptosome formation was assessed. Using immunoprecipitation in 293T cells overexpressing the PANoptosome components and NLRP12 and NLRC5, it was observed that both NLRP12 and NLRC5 could be pulled down with NLRP3, ASC, caspase-8, and RIPK3. These findings indicate that NLRP12 and NLRC5 form a cell death-inducing PANoptosome complex that contains the inflammasome along with caspase-8 and other cell death molecules to execute cell death in response to heme plus PAMPs.

### Example 9: NLRP12 and NLRC5 are Upregulated in Disease and Cause Pathology

NLRP12 and NLRC5 expression were shown to be upregulated in patients with multiple hemolytic diseases. To determine the impact of NLRP12 and NLRC5 on disease pathology, the expression of murine Nlrp12 and Nlrc5 in BMDMs was measured. Nlrp12 was significantly upregulated in response to heme plus Pam3 treatment at 36 hours post-treatment (FIG. 8), but not at 12 hours or 24 hours post-treatment. Similarly, Nlrc5 was significantly upregulated by 36 hours post-treatment with heme plus Pam3 (FIG. 8). Consistent with the increase in Nlrp12 and Nlrc5 expression, cell death began at 30-32 hours post-treatment, indicating that the expression of Nlrp12 and Nlrc5 is correlated with cell death in response to heme plus PAMPs. Moreover, Nlrp12 expression was only observed in BMDMs treated with the combination of heme plus Pam3, but not in BMDMs treated with heme or Pam3 alone (FIG. 8), indicating that heme plus PAMPs together mediate the signaling required to induce Nlrp12 and Nlrc5 expression. To further confirm these observations in human cells, single cell transcriptomics datasets from patients with hemolytic diseases (Hua et al. (2019) Blood 134:2111-2115) were analyzed. Increased expression of NLRP12 and NLRC5 was observed in erythroid, myeloid and hematopoietic stem cells in patients with beta thalassemia and SCD compared with cells derived from healthy control bone marrow. Together, these results indicate that the expression NLRP12 and NLRC5 is significantly upregulated in response to heme plus PAMPs.

In addition to hemolytic diseases, heme is known to be released during infections and inflammatory diseases due to hemorrhagic conditions and tissue damage. Since increased expression of NLRP12 was observed in hemolytic diseases, the expression profile of NLRPs in infections and pandemic diseases associated with hemorrhagic conditions was subsequently determined. Using publicly available datasets, it was found that NLRP12 was highly upregulated in patients infected with Crimean-Congo hemorrhagic fever virus (CCHFV) and macaques infected with Marburg virus. Other infections where hemolysis has been observed were also assessed, including COVID-19, which is associated with hemolytic anemia (Lazarian et al. (2020) Br. J. Haematol. 190:29-31; AbouYabis & Bell (2021) J. Hematol. 10:221-227); influenza virus infection, where the virus or hemagglutinin glycoproteins can cause hemolysis (Sato et al. (1983) Proc. Natl. Acad. Sci. USA 80:3153-3157; Huang et al. (1981) Virology 110:243-247); and pneumonia and SIRS, where hemolysis and release of free heme have been found to drive severe pathology and morbidity (Khan et al. (2009) Braz. J. Infect. Dis. 13:77-79; Wang et al. (2004) Acta Paediatr. Taiwan 45:293-295; Larsen et al. (2010) Sci. Transl. Med. 2:51ra71; Meinders & Dijkstra (2014) Blood 124:841). Using publicly available datasets, increased expression of NLRP12 was found in patients with COVID-19, influenza, pneumonia and SIRS, further implicating NLRP12 in the pathogenesis during hemorrhagic and pandemic diseases.

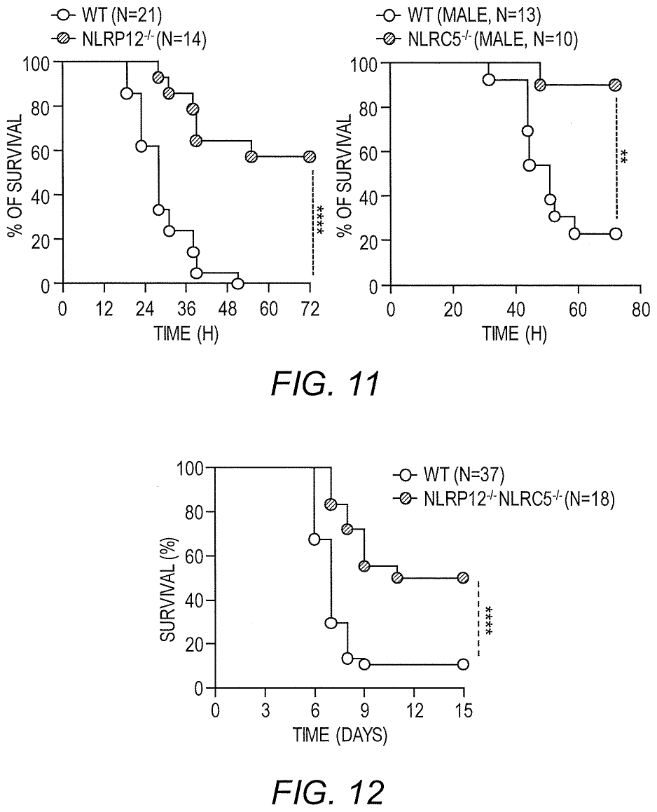

To directly test the role of NLRP12 and NLRC5 in disease pathogenesis in vivo, phenylhydrazine (PHZ), which is known to induce hemolysis (Beutler (1969) Pharmacol. Rev. 21:73-103), was injected into mice along with a non-lethal dose of LPS. In wild-type (WT) mice, an increase in the amounts of serum heme and iron was observed upon treatment with PHZ alone and the combination of PHZ and LPS, indicating that hemolysis is occurring in both groups. Moreover, levels of kidney damage markers such as blood urea nitrogen (BUN) and creatinine were significantly increased as a result of PHZ and LPS cotreatment compared to PHZ or LPS treatments alone (FIG. 9). However, there was no significant increase in the levels of liver damage marker aspartate aminotransferase (AST) in mice treated with PHZ and LPS. Previous studies have demonstrated that hemolysis and heme release can cause acute kidney injury and acute tubular necrosis (Ramos et al. (2019) Proc. Natl. Acad. Sci. USA 116:5681-5686; Vermeulen Windsant et al. (2010) Kidney Int. 77:913-920), and an increase in serum BUN was observed in WT mice treated with PHZ and LPS, but not LPS or PHZ alone (FIG. 9). To determine whether the observed pathogenesis was driven by NLRP12 or NLRC5, BUN and creatinine levels were examined in the serum from WT, Nlrp12^{-/-} and Nlrc5^{-/-} mice treated with PHZ and LPS. It was observed that serum from Nlrp12^{-/-} and Nlrc5^{-/-} mice had significantly reduced BUN and creatinine levels when compared to WT serum samples (FIG. 10). There were no significant differences in AST and iron levels in the serum between WT and Nlrp12^{-/-} mice treated with PHZ and LPS. Additionally, Nlrp12^{-/-} mice were significantly protected against PHZ and LPS-mediated mortality compared with WT mice (FIG. 11). To determine the role of NLRC5 in this pathogenesis, WT and Nlrc5^{-/-} mice were treated with PHZ and LPS and monitored for survival. It was observed that Nlrc5-mice were significantly protected from mortality in response to PHZ and LPS compared with WT mice (FIG. 11) Moreover, Nlrp12^{-/-}Nlrc5^{-/-} mice were significantly protected from mortality when infected with Plasmodium berghei ANKA (FIG. 12). Overall, these results show that hemolytic disease models, including both a ligand-based model and infection model, induce NLRP12- and NLRC5-mediated pathology, implicating NLRP12 and NLRC5 in disease pathogenesis.

What is claimed is:

1. A composition for activating or inducing the production of NLR Family Pyrin Domain Containing 12 (NLRP12) or NLR Family Caspase Activation and Recruitment Domain Containing 5 (NLRC5) comprising (a) heme and (b) at least one pathogen-associated molecular patterns (PAMP) molecule, at least one damage-associated molecular patterns (DAMP) molecule, or a combination thereof.

2. The composition of claim 1, wherein the at least one PAMP molecule comprises a triacylated lipoprotein, lipoteichoic acid, peptidoglycan, porin, zymosan, Pam₃CSK₄, diacylated lipopeptide, dsRNA, polyadenylic-polyuridylic acid, polyinosinic:polycytidylic acid, lipopolysaccharide, flagellin, single-stranded RNA, CpGA, Poly G10, Poly G3, CpG oligonucleotides, PamCysPamSK₄, Toxoplasma gondii profilin, double-stranded RNA, 5′ppp-dsRNA, phosphorylcholine, lipoarabinomannan, mycolic acid, β-1,3-glucan, N-formylmethionine, mannose-rich glycan, CL307, imiquimod, gardiquimod, resiquimod, motolimod, UC-IV150, EMD120108, IMO-2125, VTS-1463GS-9620, GSK2245035, TMX-101, TMX-201, TMX-202, isatoribine, AZD8848, MED19197, 3M-051, 3M-852, 3M-052, 3M-854A, S-34240, KU34B, CL663, SB9200, SB11285, or 8-substituted 2-amino-3H-benzo[b]azepine-4-carbozamide.

3. The composition of claim 1, wherein the at least one DAMP molecule is tumor necrosis factor α or heme metabolites.

4. A pharmaceutical composition comprising the composition of claim 1 in admixture with a pharmaceutically acceptable carrier, excipient, vehicle, diluent, or preservative.

5. A method for inducing inflammatory cell death comprising contacting cells with an effective amount of the composition of claim 1 thereby inducing inflammatory cell death.

6. A method for treating cancer or other disease or condition that would benefit from inflammatory cell death comprising administering to a subject in need of such treatment an effective amount of the pharmaceutical composition of claim 4 thereby treating the subject's cancer or other disease or condition that would benefit from inflammatory cell death.

7. The method of claim 6, wherein the other disease or condition is an infection, proliferative condition or condition comprising damaged cells.

8. A method of treating or ameliorating NLRP12-mediated or NLRC5-mediated inflammation associated with a hemolytic disease, infectious disease, inflammatory syndrome or cancer comprising administering to a subject in need thereof an effective amount of an inhibitor of NLRP12 or NLRC5 production or activation thereby treating or ameliorating the subject's NLRP12-mediated or NLRC5-mediated inflammation associated with the hemolytic disease, infectious disease, inflammatory syndrome or cancer.

9. The method of claim 8, wherein the inhibitor of NLRP12 or NLRC5 production or activation is a small molecule, peptide, antisense oligonucleotide, guide RNA, shRNA, antibody or an antibody fragment that directly inhibits the expression or activity of NLRP12 or NLRC5.

10. The method of claim 8, wherein the inhibitor of NLRP12 or NLRC5 production or activation is a small molecule, peptide, antisense oligonucleotide, guide RNA, shRNA, antibody or an antibody fragment that targets an upstream regulatory molecule of NLRP12 or NLRC5.

11. The method of claim 8, wherein the hemolytic disease is beta thalassemia, hemolytic anemia, or sickle cell disease; the infectious disease is SARS-CoV-2, influenza, malaria, or pneumonia; and the inflammatory syndrome is neurological inflammation or systemic inflammatory response syndrome.

12. A method for identifying an agent that inhibits NLRP12-dependent or NLRC5-dependent inflammasome, PANoptosome and inflammatory signaling production or activation and inflammatory cell death comprising
(a) contacting a sample of cells with the composition of claim 1 to produce or activate NLRP12 or NLRC5; and
(b) contacting the sample of cells of (a) with at least one test agent, wherein a decrease in the production or activation of NLRP12 or NLRC5 in the presence of the test agent as compared to production or activation of NLRP12 or NLRC5 in the absence of the test agent is indicative of an agent that inhibits NLRP12-dependent or NLRC5-dependent inflammasome, PANoptosome and inflammatory signaling activation and inflammatory cell death.

13. The method of claim 12, wherein the production or activation of NLRP12 or NLRC5 is determined by measuring the mRNA or protein amounts of NLRP12 or NLRC5; the cleavage of one or more of gasdermin D, gasdermin E, caspase-1, caspase-8, caspase-3, caspase-7; phosphorylation of mixed-lineage kinase domain-like protein; cell death; or release of IL-1β or IL-18 by the cells of the sample.


SEQUENCE LISTING (6 sequences)

SEQ ID NO: 1 - DNA, 21 bp, synthetic construct
  source 1..21: mol_type = other DNA
aagaccgcaatgcacgattag

SEQ ID NO: 2 - DNA, 20 bp, synthetic construct
  source 1..20: mol_type = other DNA
tggagcgttcccactctaca

SEQ ID NO: 3 - DNA, 20 bp, synthetic construct
  source 1..20: mol_type = other DNA
ggctgtattcccctccatcg

SEQ ID NO: 4 - DNA, 22 bp, synthetic construct
  source 1..22: mol_type = other DNA
ccagttggtaacaatgccatgt

SEQ ID NO: 5 - DNA, 21 bp, synthetic construct
  source 1..21: mol_type = other DNA
gtgccaaacgtccttttcaga

SEQ ID NO: 6 - DNA, 21 bp, synthetic construct
  source 1..21: mol_type = other DNA
agtgaggagtaagccatgctc